(19) United States
(12) Patent Application Publication
(10) Pub. No.: US 2013/0211138 A1
(43) Pub. Date: Aug. 15, 2013

(54) Removal of Contaminants from Liquid-Liquid Extraction Solvent

(71) Applicants: G. Peter van Walsum, Orono, ME (US); Adriaan Reinhard Pieter van Heiningen, Orono, ME (US); Joseph Genco, Orono, ME (US)

(72) Inventors: G. Peter van Walsum, Orono, ME (US); Adriaan Reinhard Pieter van Heiningen, Orono, ME (US); Joseph Genco, Orono, ME (US)

(73) Assignee: University of Maine System Board of Trustees, Bangor, ME (US)

(21) Appl. No.: 13/695,176

(22) PCT Filed: Apr. 28, 2011
(86) PCT No.: PCT/US11/34317

§ 371 Date: Jan. 9, 2013

Related U.S. Application Data
(60) Provisional application No. 61/329,180, filed on Apr. 29, 2010.

(51) Int. Cl. C07C 51/48

(52) U.S. Cl. CPC C07C 51/48
USPC 562/608; 252/364

(57) ABSTRACT

A method of treating a solvent in a wood processing operation is described. A first organic compound is extracted from an aqueous solution with a solvent. Then, the solvent is contacted with an alkali solution to remove a second organic compound from the solvent. For example, the first organic compound may be acetic acid and the second organic compound may be lignin. In another embodiment, a liquid-liquid extraction solvent used to extract acetic acid from an aqueous solution is rejuvenated by contacting the solvent with an alkali solution to remove a second organic compound from the solvent. The rejuvenated solvent is then recycled for further liquid-liquid extraction.

### CROSS-REFERENCE TO RELATED APPLICATIONS

This application claims the benefit of U.S. Provisional Application No. 61/329,180, filed Apr. 29, 2010, the disclosure of which is incorporated herein by reference.

### BACKGROUND OF THE INVENTION

This invention relates in general to chemical processes and in particular to processes for producing useful materials from wood.

Aqueous extraction of wood using either water or a solution of water and pulp mill liquors extracts hemicellulose sugars and acetic acid, an organic acid, from wood. These extracts also contain lignin and other compounds present in the wood. Recovery of the acetic acid and other components from this extraction liquor is economically attractive because they can be sold as commodity and value-added chemicals.

In addition to the acetic acid contained in the wood extract liquor, additional organic acid products can also be generated in the liquor through fermentation or chemical conversions. Some examples of such products include formic acid, lactic acid, butyric acid, itaconic acid or levulinic acid, to name a few.

One means of recovering organic acids from aqueous solutions is liquid-liquid extraction. One configuration of liquid-liquid extraction is to contact the aqueous phase with a solvent that will remove organic acid from the aqueous phase. The solvent and aqueous phases are immiscible and have different densities, so are separated after contact by decanting or centrifuging. The solvent phase is then distilled and the organic acid is recovered separate from the solvent. The solvent is then recycled back to the liquid-liquid extraction step to be contacted with fresh aqueous solution. The process is repeated continuously.

There are several solvents that can be used to extract acetic acid. For example, useful high-boiling compounds include trialkylamines such as trioctylamine, and trialkylphosphine oxides such as trioctylphosphine oxide (TOPO), and useful low-boiling compounds include isopropyl ether, triethylamine and ethyl acetate.

It would be desirable to provide improvements in these wood processing operations and other chemical processes.

### SUMMARY OF THE INVENTION

This invention relates to a method of treating a solvent in a wood processing operation. A first organic compound is extracted from an aqueous solution with a solvent. Then, the solvent is contacted with an alkali solution to remove a second organic compound from the solvent. For example, the first organic compound may be acetic acid and the second organic compound may be lignin.

In another embodiment, the invention relates to a method of rejuvenating a liquid-liquid extraction solvent. A liquid-liquid extraction solvent used to extract acetic acid from an aqueous solution is rejuvenated by contacting the solvent with an alkali solution to remove a second organic compound from the solvent. For example, the second organic compound may be lignin or another contaminant. The rejuvenated solvent is then recycled for further liquid-liquid extraction.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a process flow diagram of liquid-liquid extraction, distillation and alkaline back extraction for integration of organic acid recovery according to the invention into an alkaline wood pulping process.

FIG. 2 is plot comparing the results of distillation-recycled extract on hydrolyzed and non-hydrolyzed hemicellulose extract, with and without washing (respectively) with 1M NaOH solvent.

FIG. 3 is a bar graph showing the lignin concentration before and after hydrolysis, liquid-liquid extraction and washing with NaOH.

### DETAILED DESCRIPTION OF THE PREFERRED EMBODIMENT

As described above, a liquid-liquid extraction solvent can be used to extract acetic acid or other organic acids from an aqueous solution in a wood processing operation. Solvents that will extract organic acids from aqueous solution will also extract lignin. Investigation of one such solvent, a mixture of TOPO and undecane (C₁₁H₂₄), found that the lignin was not released through a subsequent distillation step. Furthermore, as the solvent was recycled through repetitions of the extraction and distillation process, the lignin accumulated in the solvent to the point that the solvent was no longer effective at extracting organic acids.

To address this problem, the present invention provides a process for removal of lignin and other contaminants from recycled liquid-liquid extraction solvent in a wood processing operation.

More generally, the invention provides a method of treating a solvent useful in a wood processing operation. The solvent can be any solvent that is capable of extracting an organic compound from an aqueous solution, and more particularly, any solvent capable of extracting organic compounds from an aqueous wood extraction liquor. More particularly, the method can treat any solvent capable of extracting organic acids and lignin-derived compounds from an aqueous wood extraction liquor. In certain embodiments, the solvent is recycled during wood processing. Also, in certain embodiments, the solvent is an organic solvent.

For example, the solvent may be a high-boiling temperature liquid that contains compounds such as: long carbon chain trialkylamines or trialkylphosphine oxides and may contain diluents such as high boiling point alcohols (e.g., octanol) or alkanes (e.g., undecane). Alternatively, the solvent may be a low-boiling temperature liquid that contains compounds such as isopropyl ether, ethyl acetate, ethyl lactate, or short carbon chain trialkylamines such as triethylamine.

In the present method, the solvent is treated by contacting it with an alkali solution to remove an organic compound such as lignin or other contaminant from the solvent. Any suitable alkali solution can be used, such as an aqueous alkali solution, and more particularly an aqueous solution including an alkali metal salt such as an alkali metal hydroxide.

In certain embodiments, the method uses an aqueous alkali solution derived from a wood processing operation such as a pulping process. For example, Kraft pulp mills circulate several high-pH solutions through the pulping process, including white liquor, oxidized white liquor, green liquor and black liquor. Of these, white liquor, oxidized white liquor and green liquor are promising sources for a high pH solution that will remove lignin from the solvent used for liquid-liquid extraction. Using white or green liquor to remove lignin from the extraction solvent makes use of an existing, recycled process stream in the pulp mill to enable the recovery of a new valuable product. Integration of the lignin removal step with existing pulp operations also provides opportunities for additional process improvements. For example, if the wood extraction step is performed using green or white liquor, the same liquor used to remove lignin from the solvent could also be used for the wood extraction step. This could result in enhanced recovery of the desired product, since the organic acid not recovered through the distillation step would then be recycled back to the start of the process. Thus, in certain embodiments, the alkali solution used to remove the second organic compound is the same as or similar to the solvent used to extract the first organic compound.

The alkali solution can have any suitable pH. In certain embodiments, the alkali solution has a pH of about 8 or higher, more particularly about 10 or higher, and in certain embodiments about 11 or higher. The alkali solution can have any suitable composition and strength. The concentration of the alkali may be increased or decreased depending on the concentration of the lignin or other contaminant in the solvent. In one example, the method uses a strong sodium hydroxide solution having a concentration of about 1M NaOH in water. The composition of the alkali solution may also be varied depending on the boiling point of the solvent and/or the boiling point of the organic acid being removed.

The method of treating the solvent can be used to remove any functionalized organic compound of interest from the solvent. In certain embodiments, the organic compound is a phenolic compound, an aromatic compound, an organic acid, or a mixture of different compounds. In certain particular embodiments, the organic compound is lignin (a phenolic compound). Also, in certain embodiments, the organic compound removed is considered a contaminant in the solvent, so that removal of the organic compound rejuvenates the solvent to prepare it for recycling.

The concentration of lignin or other organic compound in the solvent may reach a steady level through recycling of the solvent. For example, the concentration of lignin in the extraction solvent may reach a steady level of about 2 g/L through recycling. This level is usually lower when higher pH alkaline solution is used in the method, and higher when lower pH alkaline solution is used.

Contacting the solvent with the alkali solution to remove the organic compound can be done by any suitable method. In a particular embodiment, an alkali solution is used to remove the lignin from the solvent after distillation and prior to reuse in an acid extraction step integrated with a wood pulping process. The lignin removal may be done by liquid-liquid extraction between the organic phase and the alkali solution, and the two phases are then separated by decanting or centrifuging.

In certain embodiments, the organic compound removed from the solvent is recovered as a product. Any product of interest from such a process can be recovered. For example, the product may be a high boiling point organic compound and/or a high boiling point wood extractive compound (having a BP of at least about 160° C.).

In a related embodiment, the invention provides a method of rejuvenating a liquid-liquid extraction solvent before recycling the solvent. The liquid-liquid extraction solvent can be used for extraction in any process, including but not limited to a wood processing operation. Other applications could include, for example, extracting compounds of interest from fermentation broths, food processing streams or waste water streams. The method rejuvenates a liquid-liquid extraction solvent used to extract acetic acid from an aqueous solution. The solvent is contacted with an alkali solution to remove a second organic compound from the solvent. The rejuvenated solvent is then ready to be recycled for further liquid-liquid extraction.

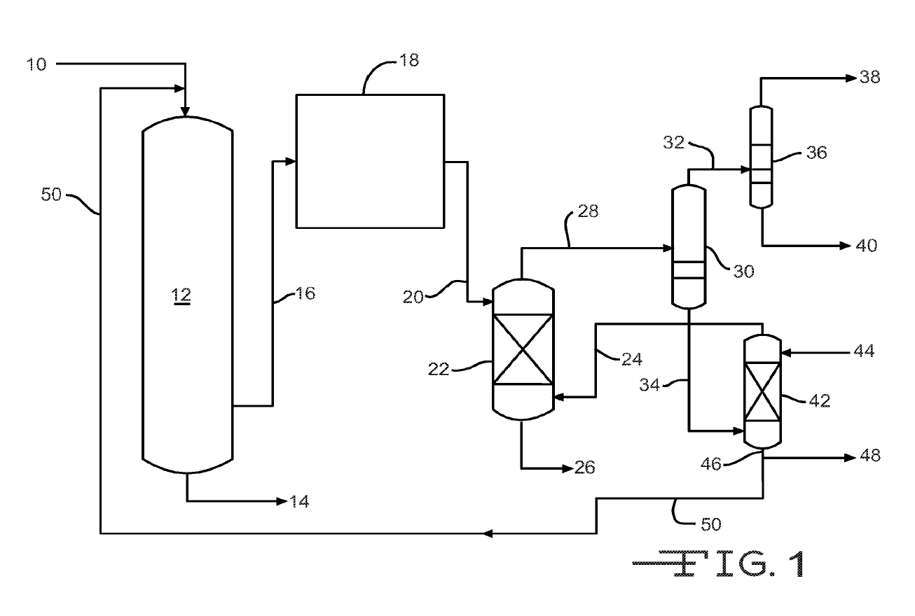

FIG. 1 is a process flow diagram showing an example of liquid-liquid extraction, distillation and alkaline back extraction for integration of organic acid recovery according to the invention into an alkaline wood pulping process. Starting at the upper left corner of the figure, a slurry 10 of wood chips and water is flowed to a wood chip extractor 12. The wood chip extractor 12 produces wood chips 14 and an aqueous extract 16 containing hemicellulose sugars and organic acid such as acetic acid. The wood chips 14 are flowed from the extractor 12 to a pulping process.

The aqueous extract 16 may be processed at 18, possibly including pH reduction, concentration, hydrolysis, precipitation, fermentation or other conversion(s). The aqueous stream 20 after processing may include organic acid, acid-soluble lignin, and other organics. This stream 20 flows to a liquid-liquid extractor 22. A clean solvent 24 also flows to the liquid-liquid extractor 22. Liquid-liquid extraction of the stream 20 with the solvent 24 in the extractor 22 produces a hemicellulose extract 26 having a reduced concentration of organic acid and acid-soluble lignin.

The liquid-liquid extraction also produces a contaminated solvent stream 28 containing the solvent, organic acid, acid-soluble lignin and other organics. The contaminated solvent stream 28 flows to a volatiles separation vessel 30. In the volatiles separation vessel 30, the contaminated solvent stream 28 is separated into an organic acid stream 32 and a lignin-containing solvent stream 34. The organic acid stream 32 can be purified at 36, for example, separated into organic acids 38 and furfural 40.

The lignin-containing solvent stream 34 is treated in a solvent cleaning vessel 42 according to the method of the invention. Green liquor, white liquor or oxidized white liquor 44 is flowed into the solvent cleaning vessel 42 into contact with the lignin-containing solvent stream in the vessel. The solvent cleaning process at 42 produces the clean solvent 24 which is recycled to the liquid-liquid extractor 22. The solvent cleaning process at 42 also produces a stream 46 of green liquor, white liquor or oxidized white liquor. A portion of this stream 46 can flow in a stream 48 to the wood pulping process. The other portion of this stream 46 can flow in a stream 50 to the beginning of the process and enter the wood chip extractor 12 along with the slurry 10 of wood chips and water.

Thus, the process flow diagram in FIG. 1 illustrates the integration of green liquor, white liquor or oxidized white liquor as solvent cleaning agents into liquid-liquid extraction and recovery of organic acids from aqueous wood extracts.

### EXPERIMENTS

Following are experimental methods and results related to solvent washing a liquid-liquid extraction solvent to remove lignin using an aqueous sodium hydroxide solution.

Materials and Methods:

Preparation of Wood Extracts:

Northeast hardwood chips were immersed and cooked in green liquor to obtain a green liquor extract that could be used for testing liquid-liquid solvent extraction. The hemicellulose extract was prepared by using a 60 L rotating digester at the University of Main Pilot Plant. The digester was loaded with 7 kg (oven dry weight) of fresh chips sized between 16 and 22.6 mm and mixed with green liquor and water (including wood moisture) at an overall liquor to wood ratio of 4:1 L/kg. For the extracts studied in this work, the green liquor charges used were from 1.44 to 3%. This system was agitated (2 rpm) at 160° C. for 110 minutes yielding an H-factor 800 hrs. The reactor was then cooled below 100° C. The free-draining liquor was collected and used for the acetic acid extraction experiments.

Acid Hydrolysis Using Dilute Sulfuric Acid:

The liquor from the wood extraction process was in some cases subjected to an acid hydrolysis step to convert oligomeric sugars to monomers. Concentrated sulfuric acid (72% w/v) was added to the hemicellulose extract to give a final concentration of 40 g/L of sulfuric acid in the solution, which reduces the pH to approximately 1. Then the solution was autoclaved for 1 hour and at 121° C.

Filtration:

After the acid hydrolysis the hemicellulose extract was filtered using filter paper (Whatman, Grade 934-AH, 1.5 μm pore size) to separate the precipitated solid lignin from the solution. About 0.4% of lignin (out of total mass of hemicellulose extract) could be separated by filtration as solid insoluble contents.

Solvent Extraction:

Solvent extraction was carried out at two scales, in 50 mL centrifuge tubes or in a 3 L stirred tank. For most recycle experiments, the larger system was used. For tests on solvent recycling, extraction conditions tested were: 370 g/L of TOPO in undecane, aqueous phase at pH 1, 70° C. and 1:1 volume ratio of organic to aqueous phases.

Small Scale Solvent Extraction:

For small scale extraction, 50 ml centrifuge tubes (50 mL; plug seal closure, Fisher) were used to contain 10 mL and 10 mL, or 20 mL and 20 mL, of aqueous and organic solutions, respectively. A water bath was used to heat and control the temperature for the experiments, The mixture was shaken by hand for 30 seconds and heated at 70° C. for 5 min by using a water bath. This step was repeated six times. Next the mixture was centrifuged at 9000 rpm for 30 min. Samples varying from 0.1-5 mL were collected from the centrifuge tubes with pipettes. Finally, the organic and aqueous phases were separated carefully from each other by using a pipette to remove the upper phase. The aqueous phase was taken to HPLC for analysis while organic phase was taken to distillation to separate acetic acid from the organic solution.

Large Scale Solvent Extraction:

For large scale extraction, a 3 L mixing tank with a stirrer was used to mix 1 L of aqueous and 1 L of organic solutions. Then the mixture was agitated and heated for 1 hour by using a hot plate and thermometer. Next the mixture was dispensed in to 1 L centrifuge tubes and centrifuged at 9000 rpm for 1 hour. Finally, the organic and aqueous phases were separated carefully from each other by using a vacuum system. The aqueous phase was analyzed via HPLC while organic phase was taken to distillation to separate acetic acid from the organic solution.

Centrifuge:

A centrifuge was used to enhance the separation of the two phases. For the small scale experiments, the 50 mL centrifuge tubes were mounted in a Sorvall RC-6 Plus superspeed centrifuge from Thermo Electron Corporation with F13-S 14×50 rotor spinning at 9000 rpm for 30 minutes. For the large scale experiments, the same centrifuge with F9-SLC-4000 rotor and 1000 mL centrifuge bottles (Fisher Scientific model Sorvall) was used at 8000 rpm for 60 minutes.

Distillation Apparatus:

The distillation apparatus consists of a boiling flask (from 50 to 3000 mL capacity), a heating mantle to heat the solution, short neck and dimpled distillation column, condenser and distillate receiving flask.

The distillation system is used to separate acetic acid from the organic solution. The distillation ran as the follows:
1. The organic solution after liquid-liquid extraction was transferred to a round-bottom boiling flask.
2. 2-3 boiling chips were added to avoid violent boiling
3. The boiling flask was connected to a distillation column and heated gradually by using a heating mantle.
4. Cooling water (18° C.) was applied to condenser tube.
5. The receiving flask was cooled by using crushed ice in a jacket around the flask.
6. The acetic acid separated from organic solution was collected in the receiving flask after approximately 1-2 hours running time.

The recovered acetic acid from distillation might include some organics such as undecane, so a centrifuge was applied for final separation of the acetic acid in the condensate.

Solvent Washing with Sodium Hydroxide (NaOH):

When recovering acetic acid from wood extracts, some amount of lignin is transferred with the acetic acid into the organic phase. Therefore, washing with 1M of NaOH is a beneficial step to remove the soluble lignin from the organic solution. This alkaline extraction removes a large portion of the contaminating lignin components. The washing procedure consisted of mixing 1 L of the post-distillation organic solution with 1 L of 1M of NaOH in a 3 L stirred mixing tank. The mixture was agitated and heated for 1 hour at 70° C. by placing on top of a hot plate. After mixing the mixture was centrifuged at 9000 rpm. After centrifuging, the two phases were separated from each other by aspirating the upper phase off of the lower aqueous phase using a vacuum aspiration system.

Solvent Recyclability:

The recyclability of solvent was tested to determine the affect of extraction and solvent stability when recycling the solvent. A fresh organic solution with optimum extraction conditions was prepared and mixed with aqueous solutions. After extraction, the organic solution containing acetic acid was taken to the distillation system to separate acetic acid from the organic solution. After distillation (and NaOH washing if applicable), the extractions were performed again and the performance of each repeated cycle of extraction and distillation were measured.

Acetic Acid Analysis:

High performance liquid chromatography, or high pressure liquid chromatography, (HPLC) with RI detection was used to quantify acetic acid (and sugars if applicable) in the aqueous phase. A Shimadzu model LC-10AT HPLC was equipped with a Bio-Rad Aminex HPX-87H column kept at 60° C. and the eluent flow rate was set to 0.6 mL/min. of 5 mM H₂SO₄. For the analysis, 1 ml was taken from the aqueous sample and diluted 10 times by adding water. 1 ml of the diluted sample was mixed with 1 ml of internal standard and then the solution was injected into HLPC for analysis. The area ratio of unknown sample of acetic acid to internal standard was determined from the HPLC chromatogram. The concentrations of the analytes were quantified using an internal standard and a calibration curve prepared with known concentrations.

Lignin Analysis:

Lignin in the aqueous and organic phases was quantified using a UV-vis spectrophotometer. The Nicolet Evolution 100 UV-Vis spectrophotometer, from Thermo Electron Corporation, was set to measure absorbance at 280 nm with respect to a standard calibration curve. About 2 mL of aqueous or organic solution was placed into a quartz macro cell (Cuvette, 10 mm, from Agilent Technologies). Next, the cell was put inside UV device and the analysis was run at 280 nm. The concentration of lignin in the solution was calculation by using the equation below:

$$C = \frac{A}{e \times L}$$

Where A is lignin absorption by UV
e is specific absorption (g/L.cm)=20 for hardwood and 24 for softwood
C is lignin concentration (g/L)
L is light path length (cm) (absorption tub width)=1 cm

### Experimental Results

When operating with samples of wood extract, recycling the solvent after extraction and solvent distillation resulted in accumulation of lignin in the solvent phase. Two methods were applied to diminish this accumulation of lignin in the organic phase. First, hydrolyzing the hemicellulose solution at low pH and removing the precipitated lignin sent less lignin on to the liquid-liquid extraction step. Second, the solvent was back extracted, or “washed,” with an aqueous solution of sodium hydroxide after the distillation step.

Washing Solvent:

In the case of extracting the hemicellulose system, table 1 shows the effects on lignin concentration of washing the organic solution with deionized water. In this case, relatively little lignin is transferred out of the organic phase and into the aqueous phase. Table 2 shows results from washing the solvent with 1 molar NaOH. With the NaOH wash, most of the lignin is removed from the organic phase.

TABLE 1
Organic solution after distillation and back-extraction by deionized water

| UV Analysis for Lignin: | Lignin (g/L): |
|---|---|
| Organic before washing | 10.5 |
| Organic after washing | 9.9 |
| Water after washing | 0.6 |

TABLE 2
Organic solution after distillation and back-extraction by 1M NaOH

| UV Analysis for Lignin: | Lignin (g/L): |
|---|---|
| Organic before washing | 4.5 |
| Organic after washing | 1.2 |
| NaOH solution after | 3.3 |
| washing | |

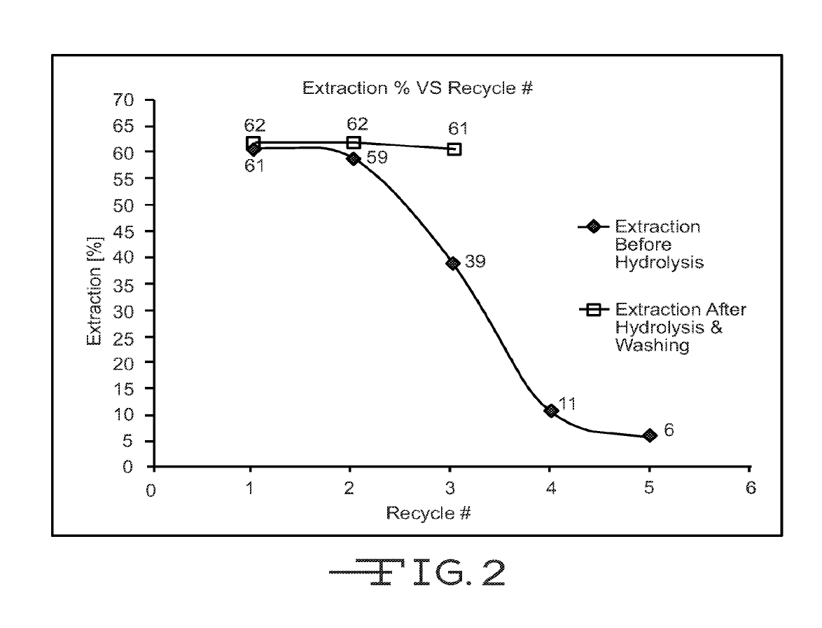

FIG. 2 shows a comparison of results for recycling solvent in 2 different systems. One system involved solvent extraction of wood extract and recycling the solvent after distillation for use in a subsequent solvent extraction. The second system involved solvent extraction of acid hydrolyzed wood extract from which acid precipitated lignin had been removed, and then recycling the solvent through distillation and a washing step using 1M NaOH. The results are for green liquor hardwood extract system and TOPO in undecane. As can be seen in FIG. 2, for the unwashed solvent, the acetic acid extraction efficiency diminishes sharply with recycling of the solvent. For the system using acid hydrolyzed material and the sodium hydroxide wash maintains its extraction efficiency through the recycles.

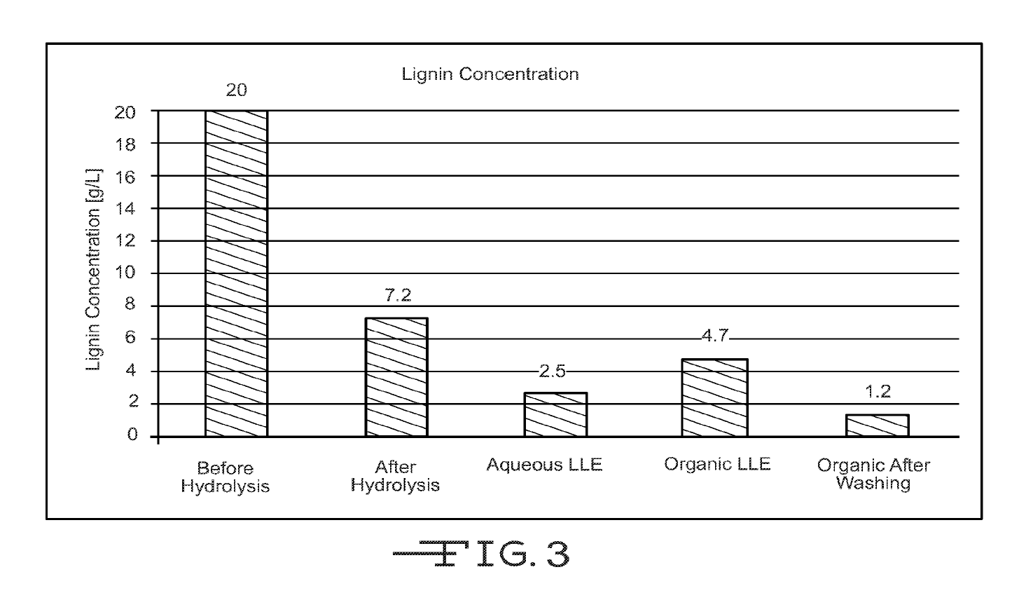

FIG. 3 shows the lignin concentration before and after hydrolysis, liquid-liquid extraction and washing with NaOH. As seen in the figure, the initial concentration of lignin in green liquor extract was 20 g/L. It was reduced to 7.2 g/L after doing acid hydrolysis and filtration of acid-precipitated lignin. Then about 65% (4.7 g/L) of the lignin in the aqueous phase transferred to the organic phase through the one stage of liquid-liquid extraction. Finally, about 73% (3.5 g/L) of lignin was removed from the organic solution by washing with 1M NaOH solution.

1. A method of treating a solvent in a wood processing operation comprising:
extracting a first organic compound from an aqueous solution with a solvent in a wood processing operation; and then
contacting the solvent with an alkali solution to remove a second organic compound from the solvent.

2. The method of claim 1 wherein the alkali solution comprises an aqueous solution including an alkali metal salt.

3. The method of claim 2 wherein the alkali solution comprises an aqueous alkali solution from a wood pulping process.

4. The method of claim 3 wherein the alkali solution comprises green liquor, white liquor, oxidized white liquor, or black liquor.

5. The method of claim 4 wherein the solvent also comprises green liquor, white liquor, oxidized white liquor, or black liquor.

6. The method of claim 1 wherein the alkali solution has a pH of about 8 or higher.

7. The method of claim 1 wherein the second organic compound is selected from the group consisting of phenolic compounds, aromatic compounds, organic acids, or mixtures thereof.

8. The method of claim 7 wherein the second organic compound comprises lignin.

9. The method of claim 1 wherein the solvent is recycled during the wood processing operation.

10. The method of claim 1 wherein the first organic compound comprises acetic acid.

11. The method of claim 1 wherein treating the solvent rejuvenates the solvent.

12. The method of claim 1 wherein the wood processing operation includes a wood pulping operation.

13. The method of claim 1 wherein the solvent is the same as the alkali solution.

14. The method of claim 1 wherein the second organic compound is recovered as a product.

15. The method of claim 14 wherein the product is a high boiling point organic compound.

16. The method of claim 14 wherein the product is a high boiling point wood extractive compound.

17. A method of rejuvenating a liquid-liquid extraction solvent before recycling the solvent comprising:
rejuvenating a liquid-liquid extraction solvent used to extract acetic acid from an aqueous solution, by contacting the solvent with an alkali solution to remove a second organic compound from the solvent; and then
recycling the rejuvenated solvent for further liquid-liquid extraction.

18. The method of claim 17 wherein the alkali solution comprises an aqueous solution including an alkali metal salt.

19. The method of claim 18 wherein the alkali solution comprises an aqueous alkali solution from a wood pulping process.

20. The method of claim 17 wherein the alkali solution has a pH of about 8 or higher.

21. The method of claim 17 wherein the second organic compound is selected from the group consisting of phenolic compounds, aromatic compounds, organic acids, or mixtures thereof.

22. The method of claim 21 wherein the second organic compound comprises lignin.

23. The method of claim 17 wherein the liquid-liquid extraction solvent is used in a wood processing operation.
